(19) United States
(12) Patent Application Publication
(10) Pub. No.: US 2005/0171467 A1
(43) Pub. Date: Aug. 4, 2005

(54) Multiple function surgical device

(76) Inventor: Jaime Landman, St. Louis, MO (US)

Correspondence Address: Kenneth K. Vu, 22872 Avenida Empresa, Rancho Santa Margarita, CA, 92688, US

(21) Appl. No.: 10/768,608

(22) Filed: Jan. 30, 2004

(51) Int. Cl. A61M 1/00; A61M 25/16

(52) U.S. Cl. 604/35; 604/537

(57) ABSTRACT

A surgical device providing multiple functions including aspiration, irrigation, traction, filtration, dissection and compression of tissue, the surgical device comprising an elongate shaft having a proximal end and a distal end, a mobilization tip operatively attached at the distal end of the elongate shaft for manipulating tissue, and a valve assembly operatively connected to the proximal end of the elongate shaft for selectively delivering and removing an irrigation fluid to and from a surgical site through the elongate shaft. The surgical device is operable with one hand. The surgical device is dimensioned according to its use in either open or minimally invasive surgery. The mobilization tip comprises a traction-enhancing material formed of reticulated foam or from a woven or braided fabric. The mobilization tip further includes a porous filter for preventing biological matters from being inadvertently drawn into the aspiration holes or windows at the distal end of the elongate shaft.

### BACKGROUND OF THE INVENTION

1. Field of the Invention

This invention generally relates to medical devices for use in open and minimally invasive or laparoscopic surgeries and, more particularly, to a surgical device providing multiple functions including aspiration, irrigation, traction, filtration, dissection and compression of tissue.

2. Discussion of Related Art

Surgical devices providing irrigation and aspiration of a surgical site are well known in the art. In particular, suction-irrigation devices are used to clean and clear surgical sites of contamination, blood, biological matter and/or debris during the course of open and minimally invasive surgeries as generally illustrated in FIGS. 1 and 2, respectively. In many instances, an irrigation fluid such as saline is introduced to a surgical site and then aspirated or vacuumed from the site. A suction-irrigation device typically includes an elongate tube, sized and configured to operate through a trocar in the case of minimally invasive surgery, and a valve system at the proximal end to alternately deliver and remove the irrigation fluid. The elongate tube is constructed of metal or plastic tubing having an open distal tip and a distal end portion that may include side-holes to allow suction when the distal tip is occluded. The construction of the present suction-irrigation devices is such that it limits the devices to their specific functions. In other words, the construction of the present suction-irrigation devices does not allow them to be used for other purposes or functions.

With the costs of surgery keep rising, it would be practical to provide surgeons with a device having multiple functions so as to reduce surgery time and costs. For example, it would be advantageous to provide surgeons with an instrument that would allow them to simultaneously dissect and aspirate. In another example, a surgeon may want to mobilize or move a piece of tissue from one location to another location during the course of surgery. This typically requires the use of a suction-irrigation device as discussed above and a mobilization device. The mobilization device generally comprises an elongate shaft with a handle at a proximal end and a mobilization tip or wand having a piece of traction-enhancing material at a distal end. In this instance, the surgeon would need to use both hands simultaneously to manipulate the suction-irrigation device and the mobilization device. Moreover, in the case of minimally invasive surgery, the mobilization device would require a separate trocar to be placed in the patient.

Accordingly, there is a need in the art for a surgical device that provides multiple functions including at least one of aspiration, irrigation, traction, filtration, dissection and compression of tissue. The ability for a surgical device to provide multiple functions would provide for optimal vascular control during surgery. This multiple function surgical device would not require the simultaneous use of both hands to operate and, in the case of minimally invasive surgery, would need only one trocar port to perform its functions. As a result, the multiple function surgical device reduces surgery time and costs. It is preferable that this surgical device has a distal end portion that is not subject to suction-lock.

### SUMMARY OF THE INVENTION

The present invention is directed to a surgical device capable of providing multiple functions including at least one of aspiration, irrigation, traction, filtration, dissection and compression of tissue. The multiple function surgical device comprises an elongate shaft having a proximal end and a distal end, a mobilization tip operatively attached at the distal end of the elongate shaft for manipulating tissue, and a valve assembly operatively connected to the proximal end of the elongate shaft for selectively delivering and removing an irrigation fluid to and from a surgical site through the elongate shaft. The surgical device may be operated with one hand in an open or minimally invasive surgical procedure. The surgical device is dimensioned according to its use in either open or minimally invasive surgery. The mobilization tip comprises a traction-enhancing material that may be formed of reticulated foam or from a woven or braided fabric. The mobilization tip further includes a porous filter for preventing biological matters such as loose tissues, clots, fats or other debris from being inadvertently drawn into the aspiration holes or spaces at the distal end of the elongate shaft so as to prevent suction-locking or vacuum-locking. The diameter of the elongate shaft may be reduced at the distal end so as to allow different material filter to be attached thereto.

### DESCRIPTION OF THE DRAWINGS

The accompanying drawings, which are included in and constitute a part of this specification, illustrate the embodiments of the invention and, together with the description, explain the features, advantages and principles of the invention. In the drawings:

FIG. 1 illustrates a top view of an open surgery requiring at least two instruments;

FIG. 2 illustrates a side view of a minimally invasive or laparoscopic surgical setup with multiple trocars;

FIG. 3 is a perspective view of a multiple function surgical device having a compressed tip in accordance with a first embodiment of the invention;

FIG. 4 is a perspective view of a multiple function surgical device having an expanded tip in accordance with a second embodiment of the invention;

FIG. 5 is an enlarged view of the surgical device shown in FIG. 3 including a traction-enhancing structure;

FIG. 6 is an enlarged view of the surgical device shown in FIG. 4 including a traction-enhancing structure;

FIG. 7 is a side section view of a multiple function surgical device having a reduced diameter at its distal end portion in accordance with another embodiment of the invention;

FIG. 8 is an enlarged view of the surgical device shown in FIG. 7;

FIG. 9 is a perspective view of the distal end of the surgical device shown in FIG. 7 prior to attachment of a traction-enhancing filter;

FIG. 10 is a perspective view of the distal end of the surgical device shown in FIG. 7 after attachment of the traction-enhancing filter;

FIG. 11 is a perspective view of the distal end of the surgical device shown in FIG. 7 attached with an open-end traction-filter in accordance with another embodiment of the invention; and

FIG. 12 illustrates perspective views of multiple function surgical devices having distal end portions with reduced diameters and large aspiration holes or spaces in accordance with additional embodiments of the invention.

### DETAILED DESCRIPTION OF THE INVENTION

The following detailed description refers to the accompanying drawings that illustrate the embodiments of the present invention. Other embodiments are possible and modifications may be made to the embodiments without departing from the spirit and scope of the invention. Thus, the following detailed description is not meant to limit the invention. Rather the scope of the invention is defined by the appended claims.

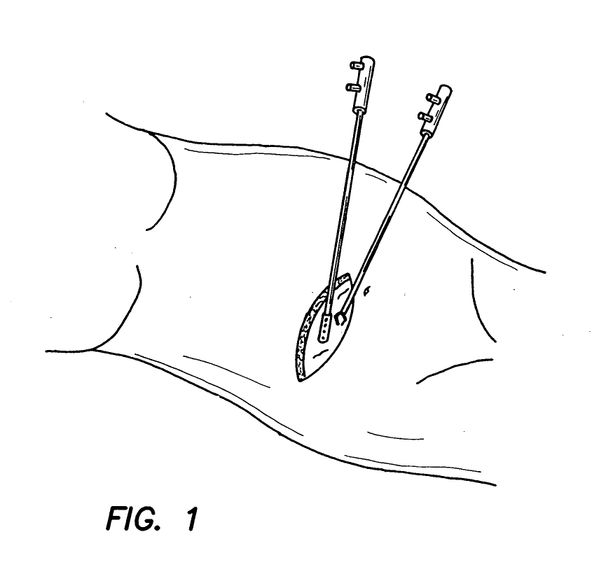

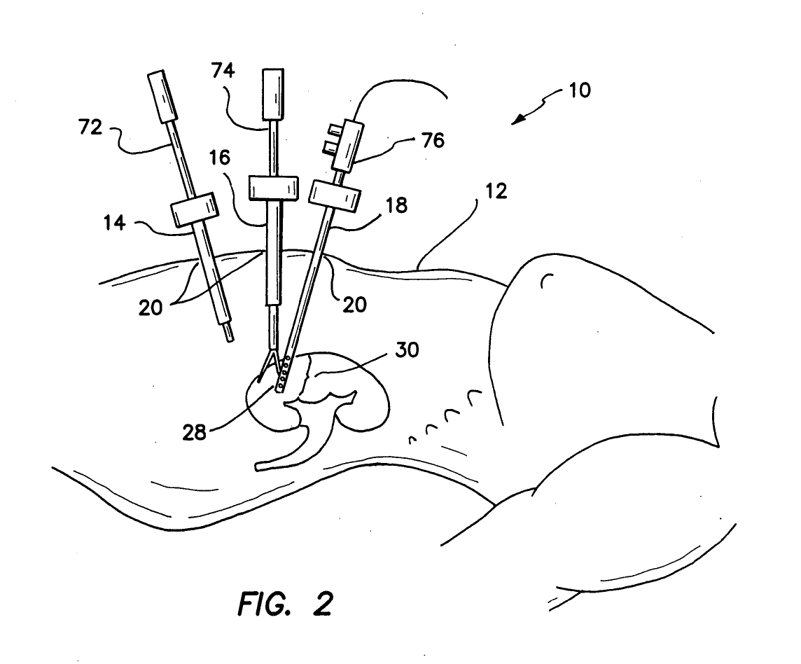

FIGS. 1 and 2 illustrate typical open and minimally invasive or laparoscopic surgeries, respectively, requiring the use of multiple instruments. In particular, FIG. 2 illustrates a side view of a typical minimally invasive or laparoscopic surgical setup 10 in a human body 12. For this surgery, three trocar ports 14, 16 and 18 are placed into a body cavity 20 such as the abdominal wall. Trocar port 14 may be used to accommodate a laparoscope 22 to view the surgical site, trocar port 16 may be used to accommodate a grasping or cutting instrument 24, and trocar port 18 may be used to accommodate a device 26 having a mobilization tip or wand. Mobilization device 26 commonly includes a traction-enhancing member 28 at a distal end portion 30. Traction-enhancing member 28 includes a traction material such as cotton, which is very absorbent and exhibits an attraction to moist surfaces. With this laparoscopic surgical setup, a surgeon may use one hand to grasp cutting instrument 24 and the other hand to grasp mobilization device 26. Similarly, during the course of an open surgical procedure as illustrated in FIG. 1, a surgeon may use one hand to grasp one instrument and the other hand to grasp another instrument. In general, most surgical procedures require the use of multiple instruments and, as such, it would be advantageous to minimize the number of instruments used during the course of a surgical procedure.

As can be seen in FIG. 2, the laparoscopic procedure makes full use of three strategically placed trocar ports 14, 16 and 18. Should another instrument such as a suction-irrigation device is required, mobilization device 26 may be removed and the suction-irrigation device may be placed in trocar port 18. That is, trocar port 18 may be used to accommodate the suction-irrigation device in place of mobilization device 26. The suction-irrigation device generally includes an elongate shaft connected to a valve at a proximal end. The elongate shaft is sized and configured to extend through trocar port 18 and into the surgical site. The valve may be connected to a fluid source and a vacuum source and operates to alternately permit the flow and suction of an irrigation fluid through the elongate shaft. The distal end of the elongate shaft is substantially open and may include side-holes. The side-holes are configured to break the suction that may occur when the open distal end is pressed against a soft, compliant, thin or loosely attached tissue. Specifically, the side-holes are designed to address the problem of vacuum-lock that may occur with common suction devices. For example, when a suction device is placed within a pool of fluid such as blood during a surgical procedure, adjacent tissues may be drawn into the open distal end resulting in a vacuum-lock. This would require the suction function to be terminated and re-started, which is time consuming. This is in addition to the time required to exchange instruments such as the exchange between the mobilization device and the suction-irrigation device. Accordingly, it is particularly useful to have instrumentation in both open and laparoscopic surgical procedures that are configured to provide more than just their traditional functions.

For example, in laparoscopic surgery, scissors and graspers are commonly connected to an electrosurgical instrument so that they can be used to coagulate severed blood vessels or to cut, electrosurgically, through tough or very vascular structures. This is advantageous since it would not be practical to exchange a mechanical grasper for an electrosurgical probe or to place another trocar into the body to accommodate the occasional use of a single instrument during a laparoscopic procedure.

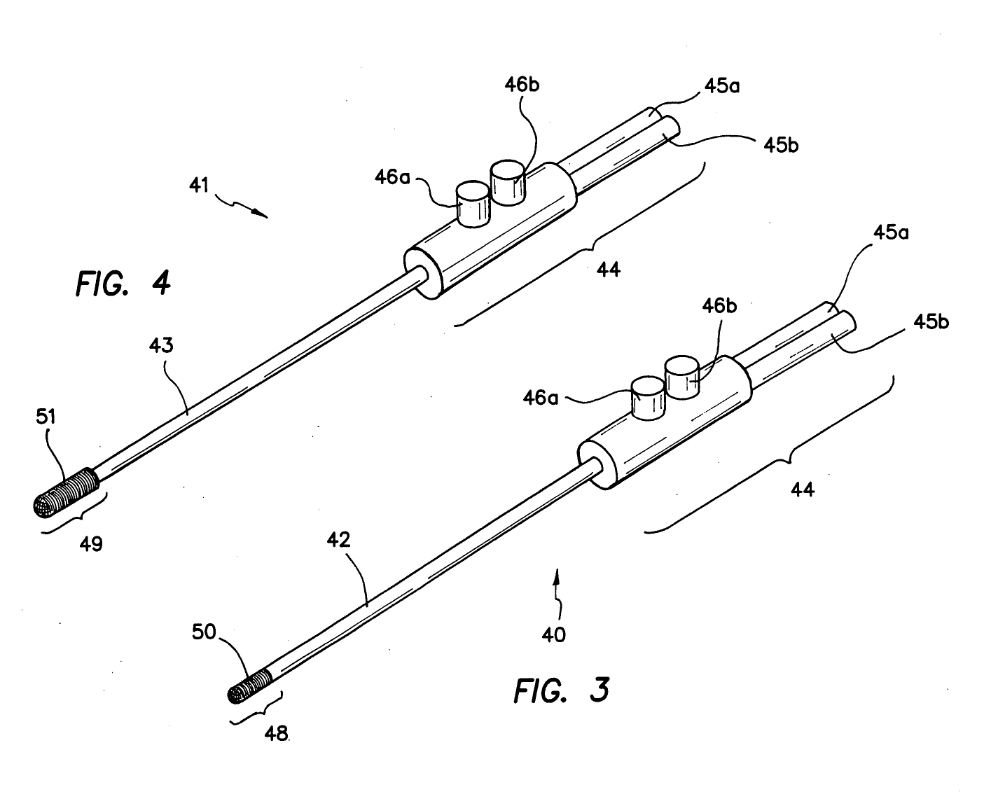

FIG. 3 is a perspective view of a multiple function surgical device 40 in accordance with a first embodiment of the invention. Surgical device 40 provides a novel multi-function instrument for performing open or minimally invasive surgeries. Surgical device 40 includes numerous features necessary for the performance of a surgical procedure such as aspiration, irrigation, traction, filtration, compression and/or dissection of tissue. The ability to perform these multiple functions simultaneously is a novel feature of the invention that is superior to any available technology in open and laparoscopic surgeries. It should be noted that surgical device 40 is dimensioned according to its use in either open or minimally invasive surgery. For example, surgical device 40 should be sized and configured to fit through a trocar port in the case of minimally invasive surgery as generally shown in FIG. 2.

Surgical device 40 includes an elongate shaft 42 and a subassembly 44 having a first connection port 45(a), a second connection port 45(b), a first valve mechanism 46(a) and a second valve mechanism 46(b). First connection port 45(a) provides a source of suction, second connection port 45(b) provides a source of irrigation, first valve mechanism 46(a) operates to actuate the source of suction through first connection port 45(a), and second valve mechanism 46(b) operates to actuate the source of irrigation through second connection port 45(b). First and second valve mechanisms 46(a) and 46(b) are preferably on/off switches in the form of trumpet valves which allow the surgeon to selectively choose the suction or irrigation features. Elongate shaft 42 further includes a distal end 48 for providing mobilization and manipulation of organs or tissues. Distal end 48 is substantially open and may include aspiration holes. Distal end 48 further includes a compressed tip and a filter 50 covering the compressed tip to prevent suction-lock when surgical device 40 is in intimate contact with vulnerable tissues. The porous nature of filter 50 prevents biological matters such as delicate or loose tissues from being drawn into the open distal end or aspiration holes of elongate shaft 42.

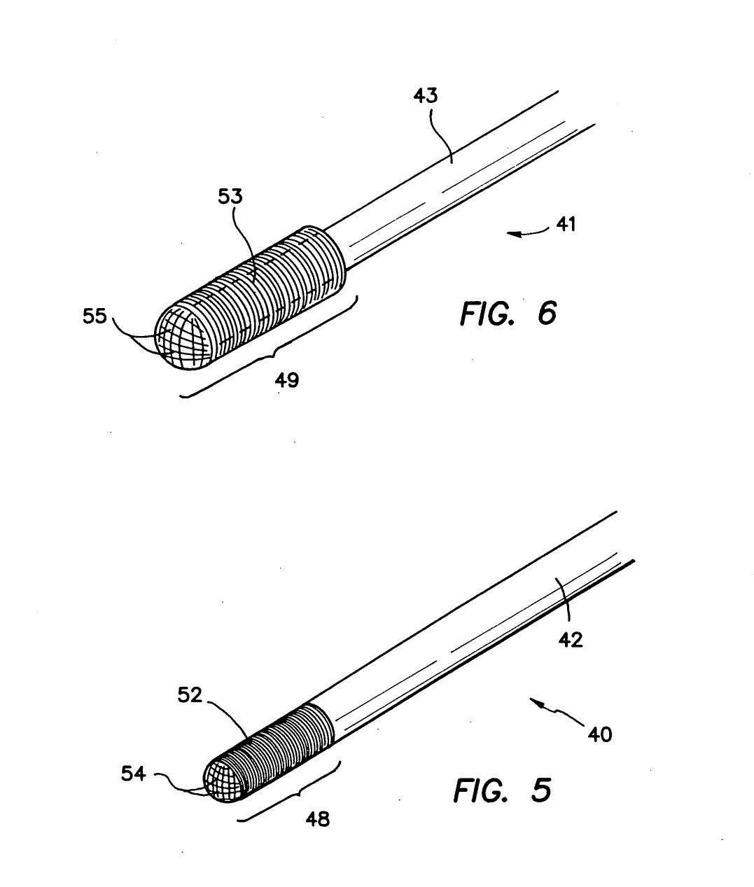

FIG. 4 is a perspective view of a multiple function surgical device 41 similar to the device 40 shown in FIG. 3 but includes an expanded distal end or tip 49 and a filter 51 covering said tip 49 in accordance with a second embodiment of the invention. In another embodiment of the invention, FIG. 5 illustrates an enlarged view of surgical device 40 as shown in FIG. 3 further including a filter 52 having a traction-enhancing structure 54 attached at distal end 48. In yet another embodiment of the invention, FIG. 6 illustrates an enlarged view of surgical device 41 as shown in FIG. 4 further including a filter 53 having a traction-enhancing structure 55 attached at distal end or tip 49. It is appreciated that the material used for traction-enhancing structures 54 and 55 would increase the efficiency of the aspiration component of surgical devices 40 and 41, respectively, as traditional suction-irrigation devices tend to become obstructed with clots, fat and/or other debris. In addition to providing filtration, the softer tips of surgical devices 40 and 41 would also make them superior for compression of bleeding tissues or vessels.

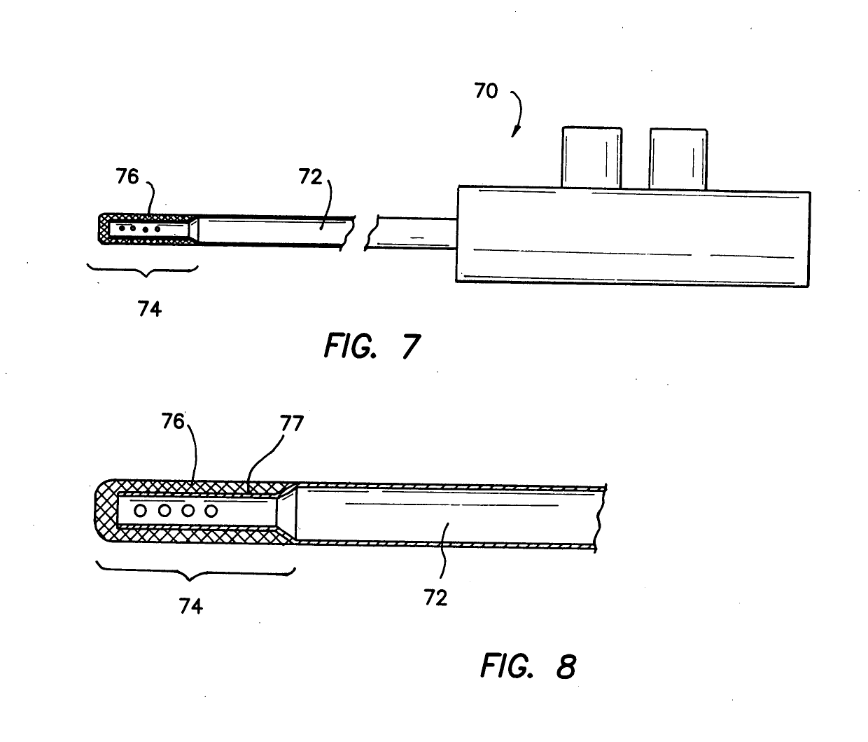

Traction-enhancing structures 54 and 55 may be formed from cotton or a cotton-like material having absorptive characteristics. Alternatively, traction-enhancing structures 54 and 55 may be formed from a reticulated or an open-cell foam or sponge. Each of traction-enhancing structures 54 and 55 may include a molded, die-cut, woven knitted or braided cover that is removably attached to distal ends or tips 48 and 49, respectively. With this configuration, the surfaces of traction-enhancing structures 54 and 55 provide a frictional component that mimics the serrations of existing surgical instruments. As illustrated in FIG. 8, a braided tubular sleeve 76 may be formed over a reticulated foam sleeve 77. Braided tubular sleeve 76 provides superior traction while foam sleeve 77 provides atraumatic flexibility and conformity to tissue irregularities or surface features. Braided tubular sleeve 76 is preferably made from a non-elastic fiber and foam sleeve 77 is preferably made from a soft, porous and elastic material. It is appreciated that braided tubular sleeve 76 maintains a tractive surface even when compressed.

As discussed above, the porous nature of filters 50, 51, 52 and 53 prevent delicate or loose tissues from entering into the openings of elongate shafts 42 and 43. Nevertheless, in the event that tissues are pressed against filters 50, 51, 52 and 53, the suction function of surgical devices 40 and 41 will automatically re-distribute through the porous filters such that devices 40 and 41 will continue to operate without interrupting the surgical procedure. As a result, surgical devices 40 and 41 can provide deep-pool suction without the complications of vacuum-locking of hidden tissues or structures. Filters 50, 51, 52 and 53 are preferably made from a porous material that allows irrigation fluid to pass from the distal ends of elongate shafts 42 and 43 to the surgical site.

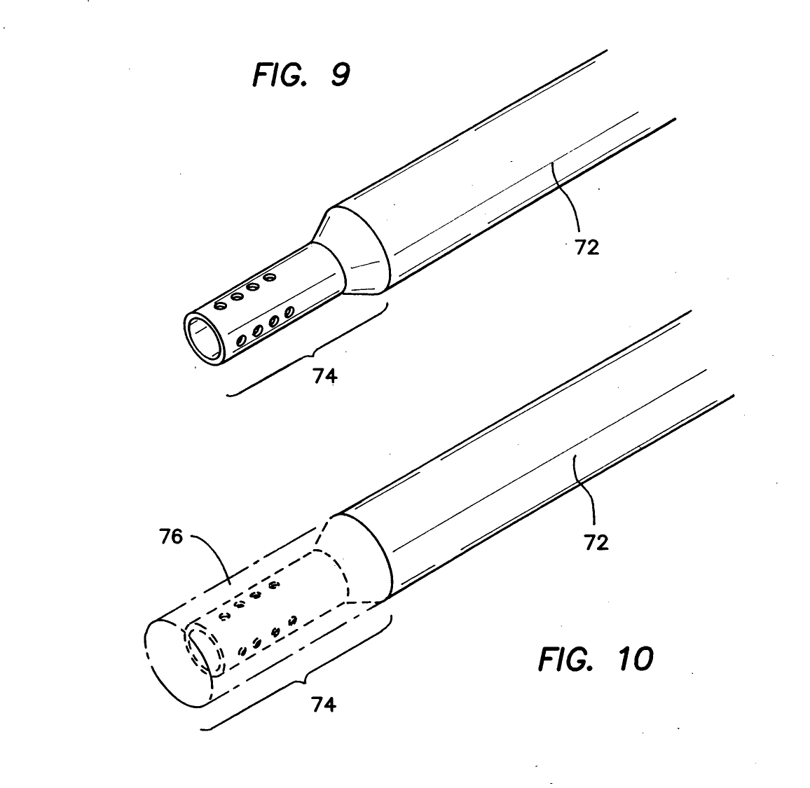

FIG. 7 illustrates a side section view of a multiple function surgical device 70 in accordance with another embodiment of the invention. Surgical device 70 includes an elongate tubular shaft 72 having a reduced diameter at its distal end portion 74. The reduced diameter allows for the placement of a traction-enhancing filter including braided tubular sleeve 76 and foam sleeve 77 without substantially increasing the diameter of the instrument shaft. For instance, a 5 mm laparoscopic suction-irrigation device is typically sized and configured to fit through a 5 mm laparoscopic trocar. If a distal attachment is placed over the shaft, the increased diameter of the suction-irrigation device may prevent it from entering or exiting the trocar. As such, the reduced-diameter of distal end portion 74 of elongate shaft 72 allows the traction-enhancing filter to be attached and still fits through a chosen trocar. FIG. 8 is an enlarged view of surgical device 70 as shown in FIG. 7. FIG. 9 is a perspective view of the distal end of surgical device 70 prior to attachment of the traction-enhancing filter. FIG. 10 is a perspective view of the distal end of surgical device 70 after attachment of the traction-enhancing filter.

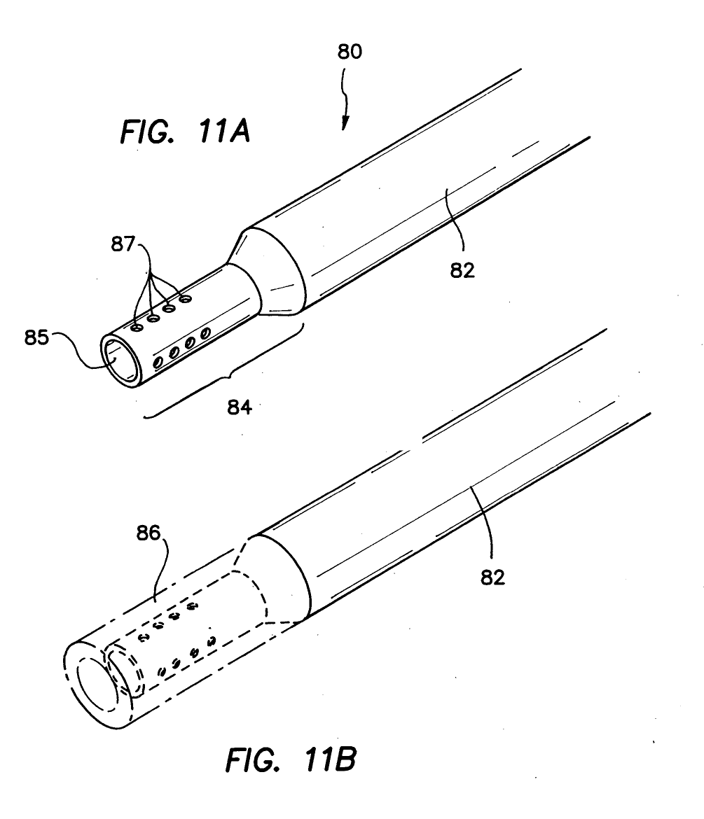

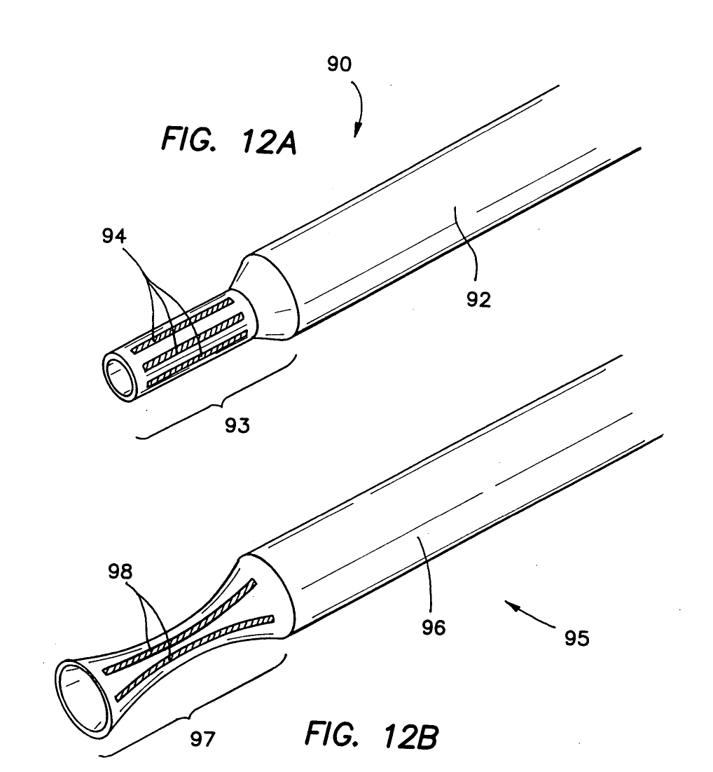

In another embodiment of the invention, FIG. 11 illustrates a perspective view of a multiple function surgical device 80 in accordance with another embodiment of the invention. Surgical device 80 is similar to the device 70 shown in FIGS. 7-10 including an elongate tubular shaft 82 having a reduced diameter at its distal end portion 84. The distal end 85 of distal end portion 84 is substantially open and may include aspiration holes 87. The reduced diameter of distal end portion 84 allows for the placement of an open-end traction-enhancing filter 86 without substantially increasing the diameter of the instrument shaft. Open-end traction-enhancing filter 86 is configured such that open distal end 85 remains open after attachment of traction-enhancing filter 86 onto distal end portion 84. In yet another embodiment of the invention, FIG. 12 illustrates perspective views of multiple function surgical devices 90 and 95 comprising elongate shafts 92 and 96 having reduced diameters at distal end portions 93 and 97, respectively. The reduced diameters at distal end portions 93 and 97 allow thicker, more absorptive and more tractive filters to be attached thereto. Moreover, distal end portions 93 and 97 include larger aspiration holes or spaces 94 and 98, respectively, that provide improved aspiration and irrigation of the surgical site.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention.

1. A surgical device comprising:
an elongate shaft defining a lumen and having a proximal end and a distal end;
a mobilization tip operatively attached at the distal end of the elongate shaft for manipulating tissue; and
a valve assembly operatively connected to the proximal end of the elongate shaft for selectively delivering and removing an irrigation fluid to and from a surgical site through the elongate shaft.

2. The surgical device of claim 1, wherein the surgical device is operable with one hand.

3. The surgical device of claim 1, wherein the mobilization tip includes a traction-enhancing material.

4. The surgical device of claim 1, wherein the mobilization tip is formed from a reticulated foam or sponge.

5. The surgical device of claim 1, wherein the mobilization tip is formed from an open-cell foam or sponge.

6. The surgical device of claim 3, wherein the traction-enhancing material includes a molded, die-cut, woven knitted or braided cover that is removably attached to the distal end of the elongate shaft.

7. The surgical device of claim 1, wherein the mobilization tip includes a foam sleeve and a braided tubular sleeve formed over the foam sleeve.

8. The surgical device of claim 7, wherein the foam sleeve is made from a soft, porous and elastic material to provide atraumatic flexibility and conformity to tissue or surface irregularities.

9. The surgical device of claim 7, wherein the braided tubular sleeve is made from a non-elastic fiber.

10. The surgical device of claim 7, wherein the braided tubular sleeve maintains a tractive surface even when compressed.

11. The surgical device of claim 1, wherein the mobilization tip includes a porous material.

12. The surgical device of claim 1, wherein the elongate shaft includes aspiration holes or spaces at the distal end.

13. The surgical device of claim 12, wherein the mobilization tip includes a porous filter for preventing a biological matter from being drawn into the aspiration holes or spaces of the elongate shaft.

14. The surgical device of claim 12, wherein the mobilization tip prevents the surgical device from suction-locking or vacuum-locking.

15. The surgical device of claim 1, wherein the valve assembly includes a first connection port providing a source of suction, a second connection port providing a source of irrigation, a first valve mechanism for actuating the source of suction through the first connection port, and a second valve mechanism for actuating the source of irrigation through the second connection port.

16. The surgical device of claim 15, wherein each of the first and second valve mechanisms comprises an on/off switch allowing an operator to selectively choose the suction or irrigation feature.

17. The surgical device of claim 1, wherein the distal end of the elongate shaft is compressed.

18. The surgical device of claim 1, wherein the distal end of the elongate shaft is expanded.

19. The surgical device of claim 1, wherein the surgical device provides multiple functions including at least one of aspiration, irrigation, traction, filtration, dissection and compression of tissue.

20. The surgical device of claim 19, wherein the multiple functions may be performed simultaneously.

21. The surgical device of claim 1, wherein the surgical device is used in an open surgical procedure.

22. The surgical device of claim 1, wherein the surgical device is used in a minimally invasive or laparoscopic surgical procedure.

23. The surgical device of claim 22, wherein the elongate shaft is sized and configured to extend through a trocar port to the surgical site.

24. The surgical device of claim 1, wherein the surgical device is dimensioned according to its use in either open or minimally invasive surgery.

25. The surgical device of claim 1, wherein the mobilization tip is made from a porous material allowing the irrigation fluid to pass from the distal end of the elongate shaft to the surgical site.

26. A surgical device comprising:
an elongate shaft defining a lumen and having a first diameter at a proximal end and a second diameter less than the first diameter at a distal end portion;
a mobilization tip operatively attached at the distal end portion of the elongate shaft; and
a valve assembly operatively connected to the proximal end of the elongate shaft for selectively delivering and removing an irrigation fluid to and from a surgical site through the elongate shaft,
wherein the mobilization tip has an outer diameter that is substantially the same as the first diameter such that the diameter of the elongate shaft remains substantially the same after attachment of the mobilization tip to the distal end portion.

27. The surgical device of claim 26, wherein the surgical device is operable with one hand.

28. The surgical device of claim 26, wherein the mobilization tip includes a traction-enhancing material.

29. The surgical device of claim 26, wherein the mobilization tip includes an open-end traction-enhancing filter.

30. The surgical device of claim 26, wherein the mobilization tip is formed from a reticulated foam or sponge.

31. The surgical device of claim 26, wherein the mobilization tip is formed from an open-cell foam or sponge.

32. The surgical device of claim 28, wherein the traction-enhancing material includes a molded, die-cut, woven knitted or braided cover that is removably attached to the distal end portion of the elongate shaft.

33. The surgical device of claim 26, wherein the mobilization tip includes a foam sleeve and a braided tubular sleeve formed over the foam sleeve.

34. The surgical device of claim 26, wherein the surgical device is dimensioned according to its use in either open or minimally invasive surgery.

35. A surgical device comprising:
an elongate shaft defining a lumen and having a proximal end and a distal end;
a mobilization tip operatively attached at the distal end of the elongate shaft; and
a valve assembly operatively connected to the proximal end of the elongate shaft for selectively delivering and removing an irrigation fluid to and from a surgical site through the elongate shaft,
wherein the surgical device provides multiple functions including at least one of aspiration, irrigation, traction, filtration, dissection and compression of tissue.

36. The surgical device of claim 35, wherein the surgical device can be used in an open or minimally invasive surgical procedure.

37. The surgical device of claim 35, wherein the surgical device is dimensioned according to its use in either open or minimally invasive surgery.

38. The surgical device of claim 35, wherein the elongate shaft includes aspiration holes or spaces at the distal end.

39. The surgical device of claim 38, wherein the mobilization tip includes a porous filter for preventing a biological matter from being drawn into the aspiration holes or spaces of the elongate shaft.

40. The surgical device of claim 38, wherein the mobilization tip prevents the surgical device from suction-locking or vacuum-locking.
